(19) United States
(12) Patent Application Publication
(10) Pub. No.: US 2026/0272438 A1
(43) Pub. Date: Sep. 17, 2026

(54) DEVICE FOR FIXING A CRANIOTOMY OPERCULUM TO THE CRANIAL VAULT TO BE INSERTED INTO THE CRANIOTOMY CUT

(71) Applicant: NTPLAST S.R.L., Roma (IT)

(72) Inventor: Igino Romolo Gazzani, Roma (IT)

(21) Appl. No.: 19/168,134

(22) PCT Filed: Mar. 18, 2024
(86) PCT No.: PCT/IT2024/050058

§ 371 Date: Sep. 23, 2025

(30) Foreign Application Priority Data
Mar. 31, 2023 (IT) 102023000006273

(51) Int. Cl. A61B 17/02

(52) U.S. Cl. CPC A61B 17/02

(57) ABSTRACT

A fixing device (10) is described for fixing a craniotomy operculum to a cranial vault (17), designed to be positioned in a craniotomy slot (22), comprising: at least one support tensor (12); first closing means (11′, 13′) operatively connected to the support tensor (12); second closing means (14) designed to be fixed to the support tensor (12) to complete its closure; at least one handle (15) removably connected to the support tensor (12) and designed to manipulate the device (10) to bring it from its rest position to its operational fixing position, wherein the first closing means (11′, 13′) are connected to a distal end of the support tensor (12) and the second closing means (14) are designed to be fixed to the support tensor (12) at a position close to the distal end of the tensor support (12), so that, when the device (10) is in its operational fixing position, the first closing means (11′, 13′) are placed against the internal distal cranial surface and the second closing means (14) are placed against the external cranial surface.

The present invention refers to a device for the stable fixation of a craniotomy operculum to the cranial vault, to be positioned along a craniotomy cut.

Craniotomy is the surgical operation to open the skull which is performed through the incision and cutting of the cranial vault. Craniotomy is a mandatory procedure for any intra-cranial surgery.

The procedure for opening the cranial vault, for performing intradural operations, involves incising the skin and subcutaneous tissue with a scalpel. Once the bone is exposed, the periosteum is incised with an electric scalpel and detached from the bone with a special instrument. Once the bone is adequately exposed, one or more holes are drilled with a special drill. After having detached the meninge underneath the holes made, an instrument called a craniotome is used, with which a free bone flap is created which completely exposes the underlying dura mater (meninge).

At this point the craniotomy is finished. At the end of the intradural procedure, the craniotomy operculum is put back in its place and secured to the free margins using special fixing devices. The periosteum is sutured again, as are the other layers above it. Obviously, between the craniotomy operculum and the bone of the cranial vault there remains fissure having the width of the blade used for cutting, the fissure being subsequently closed by the bone tissue which reconstitutes spontaneously.

The use of fixing devices of the type of those described in EP-A1-2988687 and EP-A1-3758632, in the name of the same Applicant, allows, in general, an aesthetically valid closure, preventing the craniotomy operculum from being projected, recessed, tilted or rotated.

The device described in EP-A1-2988687 essentially consists of a tensor that fits into the gap existing between the bone of the cranial vault and the craniotomy operculum, and first and second closing means suitable for fixing the craniotomy operculum in the correct position, especially without protrusions or recesses. These first closing means include:
a pair of elongated elastic arms, operatively connected to the lower part of the tensor, i.e. the part oriented towards the inside of the skull, each of the elongated elastic arms ending in two small transverse arms;
a lower base of the tensor, in turn provided with the small transverse arms which will be inserted into the epidural space (X) between the cranial vault and the dura mater.

The second closing means comprise a plate slidably mounted on the tensor, the plate being able to be pressed against the outside of the cranial vault so that fixing is obtained by tightening against the cranial bones from inside and outside, this tightening being favoured by the elasticity of the fixing device.

When the fixing device is in its operating position, the elongated elastic arms are pressed against the internal distal cranial surface and the plate is pressed against the external surface of the cranial vault.

This prior art device, while allowing optimal fixation of the craniotomy operculum, however has a drawback in that the lower base, which must be inserted into the epidural space between the dura mater and the bone of the cranial vault, is so large as to require the detachment of excessive parts of the epidural space between the dura mater and the cranial vault, a very delicate operation which involves high risks of bleeding. On the other hand, assembling the device without using the lower base, although possible, also has drawbacks due to the fact that the shaping of the elongated elastic arms are sharp at the tips with the risk of tearing, during the insertion into the epidural space, the dura mater, with high risks of bleeding.

Document US-A1-2013/282011 discloses a fixing device according to the preamble of claim 1.

The device described in document EP-A1-3758632 is a device for fixing a craniotomy operculum to a cranial vault, suitable for being positioned in a craniotomy slot, comprising:
at least one support tensor;
first closing means operatively connected to the support tensor;
second closing means suitable for being fixed to the support tensioner to complete its closure;
at least one handle removably connected to the support tensor and adapted to manipulate the closure device to bring it from its rest position to its operational fixing position, wherein the first closing means are connected to a distal end of the support tensor and the second closing means are adapted to be fixed to the support tensor in a position close to the distal end of the support tensor, so that, when the fixing device is in its operative position, the first closing means are placed against the internal distal cranial surface and the second closing means are placed against the external cranial surface, the first closing means comprising at least two elongated elastic arms operatively connected to the lower end of the tensor, each of the elongated elastic bands ending in two small elongated arms positioned transversely with respect to the elongated elastic arms; the small transversal arms have a tapered skate shape, suitable for favouring the wedging of the same small transversal arm and its subsequent sliding into the epidural space over the dura mater and the cranial vault, causing only a minimal detachment of the dura mater and reducing the risk of bleeding to a minimum.

Object of the present invention is solving the aforementioned prior art problems by providing a device for fixing a craniotomy operculum to a cranial vault, suitable for being positioned in a craniotomy slot, comprising:
at least one support tensor;
first closing means operatively connected to the support tensor;
second closing means designed to be fixed to the support tensor to complete its closure;
at least one handle removably connected to the support tensor and designed to manipulate the device to bring it from its rest position to its operational fixing position, wherein the first closing means are connected to a distal end of the support tensor and the second closing means are designed to be fixed to the support tensor in a position close to the distal end of the support tensor, such that, when the device is in its operational fixing position, the first closing means are placed against the internal distal cranial surface and the second closing means are placed against the external cranial surface,
wherein the first closing means comprises four elongated elastic arms operatively connected to the lower end of the tensor, each of the elongated elastic arms terminating in a small arm positioned transversely relative to its elongated elastic arm, wherein the four small transverse arms have a shape with a tapered slide, suitable for facilitating the wedding of the four small transverse arms and their subsequent sliding into the epidural space over the dura mater and the cranial vault, causing only a minimal detachment of the dura mater, reducing the risk of bleeding to a minimum, the small transverse arms having a contact surface with the dura mater of a substantially oval shape, and having rounded ends with a geometry designed to make them slide upon insertion into the epidural space, the elongated elastic arms also having an elliptical section.

In this way, the small transverse arms have a tapered skate shape, suitable for facilitating the wedging of the small transverse arm itself and its subsequent sliding into the epidural space over the dura mater and the cranial vault, causing only a minimal detachment of the dura mater and minimizing the risk of bleeding.

The new transverse arms will guarantee the application of the device along the craniotomy cut with a non-traumatic insertion also in correspondence with the line of the cerebral sinuses: this greatly broadens the field of application. The new geometry will avoid detaching excessive parts of the dura mater, significantly reducing the risk of bleeding.

Preferred embodiments and non-trivial variations of the present invention form the subject matter of the dependent claims.

It is understood that all attached claims form an integral part of this description.

It will be immediately obvious that countless variations and modifications can be made to what is described (for example relating to shape, dimensions, arrangements and parts with equivalent functionality) without departing from the scope of the invention as appears from the attached claims.

The present invention will be better described by a preferred embodiment, provided as a non-limiting example, with reference to the attached drawings, in which:

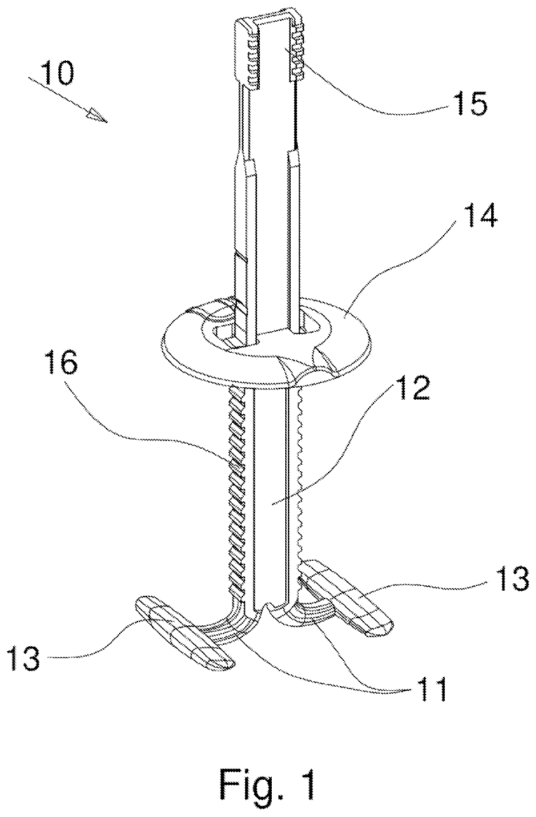

FIG. 1 shows an example of the device for stable fixation of a craniotomy operculum to the cranial vault;

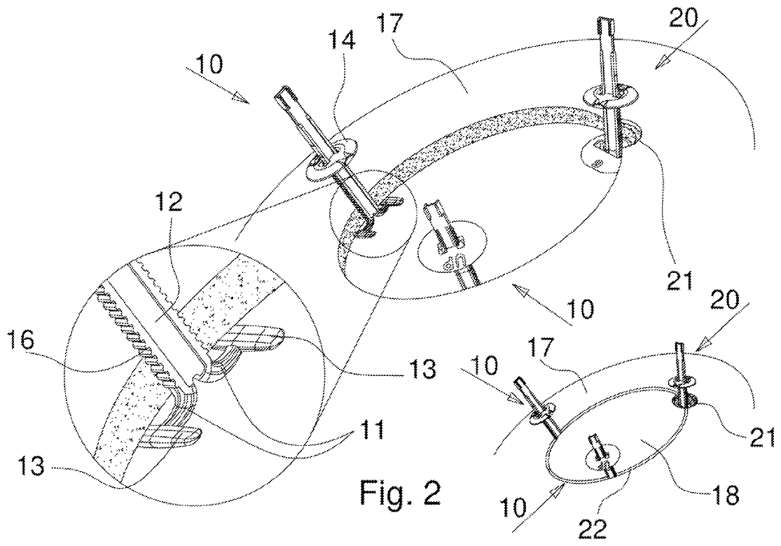

FIG. 2 shows an example of use of the device for the stable fixation of a craniotomy operculum to the cranial vault;

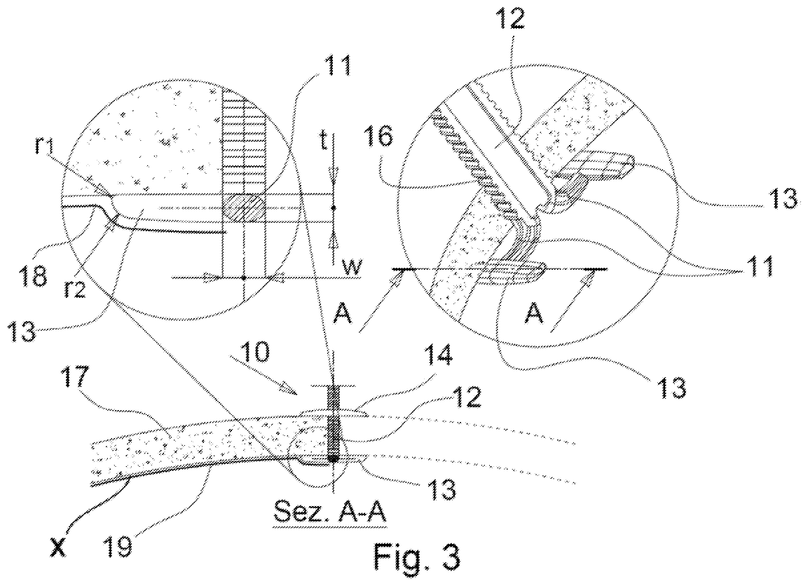

FIG. 3 shows details of the device according to the present invention; and

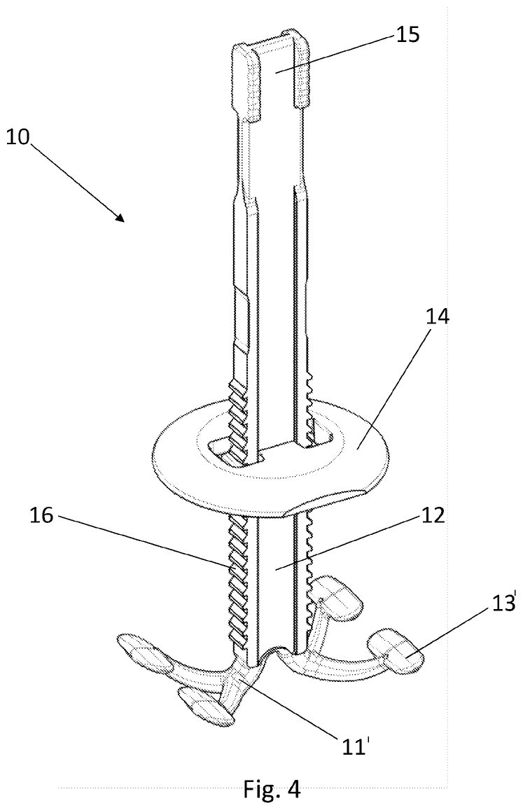

FIG. 4 shows a perspective view of a preferred embodiment of the device according to the present invention.

Referring to FIG. 1, the device for fixing a craniotomy operculum to the cranial vault, to be inserted into the craniotomy cut, is globally indicated with reference number (10). This device (10) comprises four elongated elastic arms (11′), connected to the lower part of a tensor (12) and each ending in a small elongated arm (13′) arranged transversely with respect to the axis of the elongated elastic arms (11′) , and an upper plate (14), suitable for being inserted into the upper part of the tensor (12). The tensor (12) is provided with a handle (15) in the upper part and with a plurality of fixing elements (16) (which can be, for example, small teeth as shown in the attached figures). These fixing elements (16) will be used to fix the upper plate (14) in the operational position.

Due to the elasticity of the arms (11′), the tensor (12), which connects the elastic arms (11′) and the upper plate (14), allows a fine adjustment of the position of the plate (14) depending on the thickness of the bone of the cranial vault.

The arrangement of the device (10) therefore provides that the first closing means (11′, 13′) are connected to the lower distal end of the tensor (12) and that the closing plate (14) is suitable for being fixed to the raising of the tensor (12), in a position close to the distal end of the tensor (12), such that, when the device (10) is in its operative fixing position, the first closing means (11′, 13′) are pressed against the internal distal cranial surface while the second closing means (14) are pressed against the external surface of the cranial vault.

According to a preferred embodiment, the device (10) is made of injection-moulded biocompatible elastic plastic material, for example PEEK (Polyether Ether Ketone).

FIG. 2 shows an example of use of the fixing device (10) according to the invention.

Examining in more detail the methods of use of the fixing device (10), the operator prepares the right quantity of devices (10) suitable for fixing a craniotomy operculum (18), choosing the most suitable number of devices (10), based on the size and shape of the craniotomy. In the illustrated case there are two fixing devices (10) according to the invention, to be applied to the craniotomy slot (22), and a fixing device (20), according to EP-A1-2988687, for application in correspondence with the hole (21), made for performing the craniotomy.

The operculum (18) of the bone is repositioned as shown in FIG. 2. In this way, the tensor (12) of the fixing devices (10, 20) will project outwards from the craniotomy hole (21) and the craniotomy slot (22), respectively.

Subsequently, the surgeon, after having verified the correct position of the operculum (18), pulls the tensor (12) slightly by grasping it by the handle (15), then inserts the plates (14) and tightens them against the external part of the cranial bone, thus fixing the operculum (18). In this configuration, the elastic arms (11′) are slightly flexed to be able to exert the correct push against the internal part of the skullcap.

When positioning the fixing devices (10) according to the invention, the surgeon must take care to check the correct insertion of the small transverse arms (13′) in the epidural space (X) between the dura mater (19) and the cranial vault (17), as shown in particular in FIG. 3. In this operation, it is necessary to keep the elastic arms (11′) aligned along the direction of the craniotomy slot (22) and the tensor (12) of the fixation device (10) perpendicular to the skull surface. In this way, the subsequent positioning of the craniotomy operculum (18) will take place by resting the lower surface of the operculum (18) on the small transverse arms (13′) and the subsequent tightening of the plate (14) against the external surface of the cranial vault (17) will ensure a perfect alignment of the bone flaps, without protrusions or depressions.

As regards the fixing device (20), in particular for closing the craniotomy hole (21), its use is described in patent EP-A1-2988687.

Once the fixing devices (10, 20) have been positioned, the part of the tensioner (12) that protrudes from the plate (14) after tightening is removed using a specific cutting device.

In FIG. 3, in the enlargement of Sect. A-A, one of the elastic arms (11′) is highlighted with its small transverse arms (13′) which are inserted into the epidural space (X) between the lower surface of the cranial vault (17) and the dura mater (18). As can be seen from the drawing, the small transverse arms (13′) have a tapered skate shape, suitable for facilitating the wedding of the small transverse arms (13′) and their subsequent sliding into the epidural space (X) under the dura mater (19), causing only a minimal detachment of the dura mater (19) itself, therefore reducing the risk of bleeding to a minimum.

To achieve this result, a particular shape of the elastic arms (11′) and the small transverse arms (13′) was developed. The small transverse arms (13′) have a contact surface with the dura mater (19) of a substantially oval shape, and have rounded ends with a geometry suitable for making them slide when inserted into the epidural space (X). The overall size of the lower base, made up of elastic arms (11′) and small transverse arms (13′), varies from 18.5×12.5 mm to 13′0.5×12 mm (preferable value), to reduce the dural detachment and consequently the risk of epidural bleeding.

The elastic arms (11′) have an elliptical section with the major axis (w) substantially equal to the width of the craniotomy slot (22) and the minor axis (t) substantially equal to the thickness of the small transverse arms (13′).

With this solution, a safe tightening of the inventive device (10) is permitted at truly minimal bone thickness dimensions, of the order of 0.5 mm.

The small transverse arms (13′) have a first and second radius of curvature (r1) and (r2). The first radius (r1) is very small, as it has the purpose of enabling the wedging of the small transverse arms (13′) under the dura mater (19). The second radius (r2) is much wider, as it has the purpose of facilitating the sliding of the small transverse arms (13′) under the dura mater (19).

1. A fixing device (10) for fixing a craniotomy operculum to a cranial vault (17), designed to be positioned in a craniotomy slot (22), comprising:
at least one support tensor (12);
first closing means (11′, 13′) operatively connected to the support tensor (12);
second closing means (14) designed to be fixed to the support tensor (12) to complete its closure;
at least one handle (15) removably connected to the support tensor (12) and designed to manipulate the device (10) to bring it from its rest position to its operational fixing position, in which the first closing means (11′, 13′) are connected to a distal end of the support tensor (12) and the second closing means (14) are designed to be fixed to the support tensor (12) at a position close to the distal end of the support tensor (12), so that, when the device (10) is in its operational fixing position, the first closing means (11′, 13′) are placed against the internal distal cranial surface and the second closing means (14) are placed against the external cranial surface,
wherein the first closing means (11′, 13′) comprise four elongated elastic arms (11′) operatively connected to the lower end of the tensor (12), each of the elongated elastic arms (11′) terminating in a small arm (13′) positioned transversely with respect to its own elongated elastic arm (11′), in which the four small transverse arms (13′) have a tapered skate shape, suitable for facilitating a wedging of the four small transverse arms (13′) and their subsequent sliding into the epidural space (X) over the dura mater (19) and the cranial vault, causing only a minimal detachment of the dura mater (19), reducing the risk of bleeding to a minimum, the small transverse arms (13′) having a contact surface with the dura mater (19) of substantially oval shape, and having rounded ends with a geometry designed to make them slide during insertion into the epidural space (X), the elongated elastic arms (11′) also having an elliptical section.

2. The fixing device (10) according to claim 1, wherein the overall size of the lower base, made up of elastic arms (11′) and small transverse arms (13′), varies from 18.5×12.5 mm to 13′ 0.5×12 mm.

3. The fixing device (10) according to claim 1, wherein the elliptical section of the elongated elastic arms has a major axis of length equal to the width (w) of the craniotomy slot (22).

4. The fixing device (10) according to claim 1, wherein the elliptical section of the elongated elastic arms has a minor axis of length equal to the thickness (t) of the small transverse arms (13′).

5. The fixing device (10) according to claim 1, wherein the small transverse arms (13′) have a first radius of curvature (r1) and a second radius of curvature (r2), the first radius of curvature (r1) being much smaller than the second radius of curvature (r2), the first radius of curvature (r1) configured to enable the wedging of the small transverse arms (13′) under the dura mater (19), the second radius of curvature (r2) configured to facilitate the sliding of the small transverse arms (13′) under the dura mater (19).
